(19) United States
(12) Patent Application Publication
(10) Pub. No.: US 2019/0053977 A1
(43) Pub. Date: Feb. 21, 2019

(54) CARDIOPULMONARY RESUSCITATION SYSTEM

(71) Applicant: KOHKEN MEDICAL CO., LTD., Bunkyo-ku, Tokyo (JP)

(72) Inventors: Eiichi MATSUI, Bunkyo-ku, Tokyo (JP); Mitsuo MATSUI, Bunkyo-ku, Tokyo (JP); Yukio SAITO, Bunkyo-ku, Tokyo (JP); Hitoshi TAZAKI, Bunkyo-ku, Tokyo (JP)

(21) Appl. No.: 16/079,285

(22) PCT Filed: Mar. 3, 2017
(86) PCT No.: PCT/JP2017/008422

§ 371 Date: Aug. 23, 2018

(30) Foreign Application Priority Data
Mar. 8, 2016 (JP) 2016-044363
Jun. 23, 2016 (JP) 2016-124105

(51) Int. Cl. A61H 31/00; A61M 16/00

(52) U.S. Cl. CPC A61H 31/006; A61M 16/024; A61H 2230/405; A61M 2205/05; A61M 2202/0208; A61H 2201/107; A61H 2201/5007

(57) ABSTRACT

A cardiopulmonary resuscitation system capable of performing passive oxygen administration with reduced oxygen consumption as compared with a conventional passive oxygen administration method. A cardiopulmonary resuscitation system which includes: a sternum compression unit that repeats a sternum compression cycle having, as one cycle, a compression period and a recoil period; an artificial respiration unit that repeats an artificial respiration cycle having, as one cycle, an inhalation period and an exhalation period and can supply oxygen administering gas to a patient during the recoil period; and a control means (not illustrated) that controls the artificial respiration unit and/or the sternum compression unit, and the controller judges, for each recoil period, whether supply of oxygen administering gas is required and sends a supply instruction signal to administer oxygen administering gas to the artificial respiration means unless it is judged that supply of the oxygen administering gas is not required.

### BACKGROUND

### 1. Field of the Disclosure

The present disclosure relates to a cardiopulmonary resuscitation system.

### 2. Discussion of the Background Art

As a cardiopulmonary resuscitation (also referred to as CPR) method, a method for combining sternum compression with hands and mouth-to-mouth artificial respiration is known. However, it is difficult to manually perform stable and high-quality cardiopulmonary resuscitation. Therefore, a cardiopulmonary resuscitator for automatically performing sternum compression and artificial respiration has been proposed. For example, the present applicant has proposed an automatic cardiopulmonary resuscitator for performing cardiac massage by repeatedly applying an impact at adjusted regular intervals and supplying respiratory gas for ventilation at adjusted timing and duration (see, for example, Patent Literature 1).

### CITATION LIST

### Patent Literature

Patent Literature 1: JP 2000-84028 A

### SUMMARY

### Technical Problem

Conventionally, there is an example in which a passive oxygen administration method is adopted in CPR by a method with hands. In this conventional passive oxygen administration method, oxygen is continuously supplied to a patient using an oxygen mask or the like. Here, “passive oxygen administration” means that oxygen is administered to a patient from an outside. “Passive oxygen inhalation” means that oxygen is supplied into the lungs of a patient by passive oxygen administration. In this way, by performing passive oxygen administration in addition to oxygen administration by artificial respiration while a cardiopulmonary resuscitation method is performed, it is expected to improve respiratory efficiency and to promote emission of exhaled carbon dioxide gas. However, the conventional passive oxygen administration method for continuously supplying oxygen has a problem that oxygen consumption is large. In addition, an automatic cardiopulmonary resuscitator for performing passive oxygen administration while performing CPR is not known.

An object of the present disclosure is to provide a cardiopulmonary resuscitation system for performing passive oxygen administration with reduced oxygen consumption as compared with a conventional passive oxygen administration method for continuously supplying oxygen.

### Solution to Problem

A cardiopulmonary resuscitation system according to the present invention includes: a sternum compression means that includes an impact hammer for compressing the chest of a patient and repeats a sternum compression cycle having, as one cycle, a compression period in which the impact hammer is pressed against the chest and a recoil period in which the impact hammer is separated from the chest and which be performed succeeding to the compression period; an artificial respiration means that repeats an artificial respiration cycle having, as one cycle, an inhalation period in which respiratory gas is supplied to the patient and an exhalation period in which supply of the respiratory gas is stopped and can supply oxygen administering gas for passive oxygen inhalation to the patient during the recoil period; and a control means that controls the artificial respiration means and/or the sternum compression means, wherein the control means judges, for each recoil period, whether supply of the oxygen administering gas is required and sends a supply instruction signal to administer the oxygen administering gas to the artificial respiration means unless it is judged that supply of the oxygen administering gas is not required.

In the cardiopulmonary resuscitation system according to the present invention, in a period under the following conditions (1) to (3), preferably, the control means judges that supply of the oxygen administering gas is not required and does not send the supply instruction signal. It is possible to prevent an excessive rise in respiratory tract internal pressure.

Condition (1): the entire recoil period in a case where the entire recoil period or a part of the recoil period overlaps the inhalation period

Condition (2): the entire recoil period in a case where the inhalation period starts immediately after the recoil period

Condition (3): the entire recoil period in a case where the recoil period starts immediately after the inhalation period

In the cardiopulmonary resuscitation system according to the present invention, preferably, the control means causes the sternum compression means to alternately perform an execution period in which the sternum compression cycle is executed a predetermined number of times per unit time and a standby period in which execution of the sternum compression cycle is temporarily stopped in the state of separating the impact hammer from the chest, and executes the artificial respiration cycle a predetermined number of times per unit time during the standby period. Even in a so-called synchronous mode, passive oxygen administration with reduced oxygen consumption can be performed.

In the cardiopulmonary resuscitation system according to the present invention, the control means preferably executes the artificial respiration cycle a predetermined number of times per unit time while executing the sternum compression cycle a predetermined number of times per unit time. Even in a so-called asynchronous mode, passive oxygen administration with reduced oxygen consumption can be performed.

In the cardiopulmonary resuscitation system according to the present invention, in a case where the inhalation period ends during the compression period, in the first recoil period after the end of the inhalation period, preferably, the control means judges that supply of the oxygen administering gas is not required and does not send the supply instruction signal. It is possible to more reliably prevent an excessive rise in respiratory tract internal pressure.

In the cardiopulmonary resuscitation system according to the present invention, in a case where the inhalation period starts during the compression period, in the last recoil period before the start of the inhalation period, preferably, the control means judges that supply of the oxygen administering gas is not required and does not send the supply instruction signal. It is possible to more reliably prevent an excessive rise in respiratory tract internal pressure.

In the cardiopulmonary resuscitation system according to the present invention, in the compression period overlapping with the inhalation period, the control means preferably stops pressing the impact hammer against the chest. It is possible to avoid a phenomenon (also referred to as fighting) in which the timing of sternum compression and the timing of artificial respiration are coincident with each other and exhalation of a patient and inhalation by an artificial respirator occur simultaneously.

In the cardiopulmonary resuscitation system according to the present invention, in a case where the inhalation period is started during the recoil period, the control means preferably extends the recoil period executed at the start time of the inhalation period at least until the inhalation period ends. Fighting can be avoided while the number of times of artificial respiration is secured.

In the cardiopulmonary resuscitation system according to the present invention, in a case where the inhalation period is started during the compression period and the start time of the inhalation period is in the first half period obtained by temporally dividing the compression period into two equal parts, the control means preferably hastens start of the inhalation period by the same time as the time from the start time of the compression period overlapping with the inhalation period to the start time of the inhalation period. Fighting can be avoided even in a case where the inhalation period is started during the compression period.

In the cardiopulmonary resuscitation system according to the present invention, in a case where the inhalation period is started during the compression period and the start time of the inhalation period is in the second half period obtained by temporally dividing the compression period into two equal parts, the control means preferably delays start of the inhalation period by the same time as the time from the start time of the inhalation period to the end time of the compression period overlapping with the inhalation period. Fighting can be avoided even in a case where the inhalation period is started during the compression period.

In the cardiopulmonary resuscitation system according to the present invention, it is preferable that the control means restarts the sternum compression cycle a predetermined time after the end of the inhalation period, and the sternum compression cycle restarted is started from the compression period. By setting a delay time after the end of the inhalation period, it is possible to secure time for exhalation and to prevent a respiratory tract internal pressure from becoming too high.

In the cardiopulmonary resuscitation system according to the present invention, the artificial respiration means and the sternum compression means are preferably configured as an integral device. Portability is improved, and cardiopulmonary resuscitation can be quickly started in an early stage of critical care.

In the cardiopulmonary resuscitation system according to the present invention, the artificial respiration means and the sternum compression means are preferably configured as individual devices. Cardiopulmonary resuscitation can be performed with higher accuracy.

In the cardiopulmonary resuscitation system according to the present invention, the control means is preferably mounted on a device constituting the artificial respiration means. The sternum compression means can be small and lightweight, and sternum compression can be quickly started in an early stage of critical care.

According to the present disclosure, it is possible to provide a cardiopulmonary resuscitation system for performing passive oxygen administration with reduced oxygen consumption as compared with a conventional passive oxygen administration method for continuously supplying oxygen.

### BRIEF DESCRIPTION OF DRAWINGS

FIG. 1 is a perspective view of an example of a cardiopulmonary resuscitation system according to the present embodiment.

FIG. 2 is a timing chart of a sternum compression cycle and an artificial respiration cycle in a synchronous mode, FIG. 2(a) illustrates a compression waveform, FIG. 2(b) illustrates a ventilation waveform, and FIG. 2(c) illustrates a waveform of the amount of gas supplied to a patient by FIGS. 2(a) and 2(b).

FIG. 3 is a timing chart of a sternum compression cycle and an artificial respiration cycle in a normal asynchronous mode, FIG. 3(a) illustrates a compression waveform, FIG. 3(b) illustrates a ventilation waveform, and FIG. 3(c) illustrates a waveform of the amount of gas supplied to a patient by FIGS. 3(a) and 3(b).

FIG. 4 is a first example of a timing chart of a sternum compression cycle and an artificial respiration cycle in an avoidance type asynchronous mode, FIG. 4(a) illustrates a compression waveform based on setting, FIG. 4(b) illustrates a ventilation waveform based on setting, FIG. 4(c) illustrates a compression waveform when fighting is avoided, FIG. 4(d) illustrates a ventilation waveform when fighting is avoided, and FIG. 4(e) illustrates a waveform of the amount of gas supplied to a patient by FIGS. 4(c) and 4(d).

FIG. 5 is a second example of the timing chart of the avoidance type sternum compression cycle and artificial respiration cycle, FIG. 5(a) illustrates a compression waveform based on setting, FIG. 5(b) illustrates a ventilation waveform based on setting, FIG. 5(c) illustrates a compression waveform when fighting is avoided, FIG. 5(d) illustrates a ventilation waveform when fighting is avoided, and FIG. 5(e) illustrates a waveform of the amount of gas supplied to a patient by FIGS. 5(c) and 5(d).

FIG. 6 is a third example of the timing chart of the avoidance type sternum compression cycle and artificial respiration cycle, FIG. 6(a) illustrates a compression waveform based on setting, FIG. 6(b) illustrates a ventilation waveform based on setting, FIG. 6(c) illustrates a compression waveform when fighting is avoided, FIG. 6(d) illustrates a ventilation waveform when fighting is avoided, and FIG. 6(e) illustrates a waveform of the amount of gas supplied to a patient by FIGS. 6(c) and 6(d).

FIG. 7 is a fourth example of the timing chart of the avoidance type sternum compression cycle and artificial respiration cycle, FIG. 7(a) illustrates a compression waveform based on setting, and FIG. 7(b) illustrates a ventilation waveform based on setting.

FIG. 8 is a conceptual diagram of another example of the cardiopulmonary resuscitation system according to the present embodiment.

### DETAILED DESCRIPTION OF THE PREFERRED EMBODIMENT

Next, the present invention will be described in detail by describing embodiments, but the present invention is not construed as being limited to description thereof. As long as an effect of the present invention is exhibited, the embodiments may be modified variously.

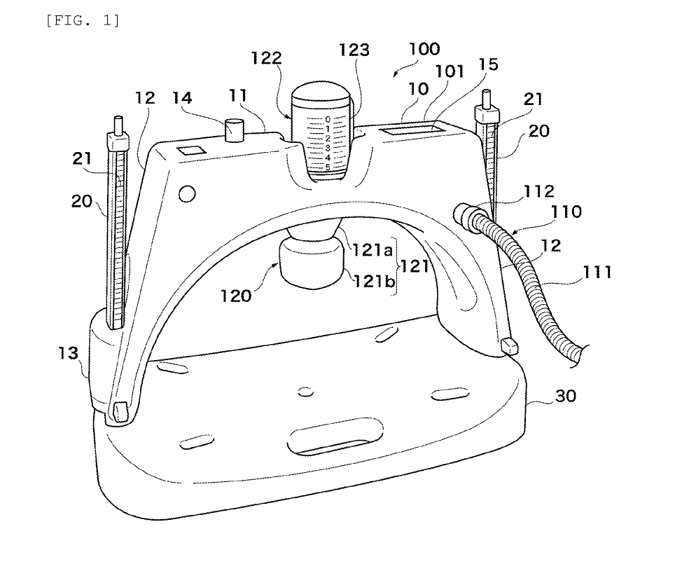

FIG. 1 is a perspective view of an example of a cardiopulmonary resuscitation system according to the present embodiment. As illustrated in FIG. 1, a cardiopulmonary resuscitation system 100 according to the present invention includes: a sternum compression means 120 that includes an impact hammer 121 for compressing the chest of a patient and repeats a sternum compression cycle having, as one cycle, a compression period in which the impact hammer 121 is pressed against the chest and a recoil period in which the impact hammer 121 is separated from the chest and which be performed succeeding to the compression period; an artificial respiration means 110 that repeats an artificial respiration cycle having, as one cycle, an inhalation period in which respiratory gas is supplied to the patient and an exhalation period in which supply of the respiratory gas is stopped and can supply oxygen administering gas to the patient during the recoil period; and a control means (not illustrated) that controls the artificial respiration means 110 and/or the sternum compression means 120, and the control means judges, for each recoil period, whether supply of the oxygen administering gas is required and sends a supply instruction signal to administer the oxygen administering gas to the artificial respiration means 110 unless it is judged that supply of the oxygen administering gas is not required.

In the cardiopulmonary resuscitation system 100, as illustrated in FIG. 1, the artificial respiration means 110 and the sternum compression means 120 are preferably configured as an integral device. The cardiopulmonary resuscitation system 100 illustrated in FIG. 1 is an automatic cardiopulmonary resuscitator equipped with an artificial respiration function and a sternum compression function. Portability is favorable, and cardiopulmonary resuscitation can be quickly started in an early stage of critical care.

The artificial respiration means 110 is an artificial respiration unit of the cardiopulmonary resuscitator 100. As illustrated in FIG. 1, the artificial respiration means (hereinafter also referred to as a first artificial respiration unit) 110 includes a hose 111 for injecting respiratory gas or oxygen administering gas into a patient and a gas supply system (not illustrated) for supplying respiratory gas or oxygen administering gas to the hose 111.

The respiratory gas or the oxygen administering gas is, for example, pure oxygen, oxygen-enriched air, or air. The respiratory gas is supplied for inhalation during the inhalation period. The oxygen administering gas is supplied for passive oxygen inhalation during the recoil period. The respiratory gas and the oxygen administering gas preferably have the same composition but may have different compositions.

One end of the hose 111 is connected to a hose insertion port 112 disposed in a housing 101 of the cardiopulmonary resuscitator 100. The other end of the hose 111 is connected to a mask (not illustrated) attached to a patient or a tracheal intubation tube (not illustrated).

The gas supply system of the first artificial respiration unit 110 includes a gas supply source such as a gas cylinder or an air tank and piping connecting the gas supply source to the hose 111. In the middle of the piping, for example, a ventilation decompressor for decreasing the pressure of driving gas to a pressure suitable for respiration, a ventilation solenoid valve for supplying respiratory gas or oxygen administering gas to the hose 111 and stopping respiratory gas or oxygen administering gas from the hose 111, and a respiratory tract internal pressure sensor for detecting the pressure in the piping between the ventilation solenoid valve and the hose insertion port 112 are disposed.

The gas supply system of the first artificial respiration unit 110 is preferably constituted by one system for respiratory gas and oxygen administering gas. In a case where the gas supply system is constituted by one system for respiratory gas and oxygen administering gas, for example, one ventilation solenoid valve is opened or closed at a predetermined timing. The gas supply system of the first artificial respiration unit 110 may be constituted by individual systems for respiratory gas and oxygen administering gas. In a case where the gas supply system is constituted by individual systems for respiratory gas and oxygen administering gas, for example, as a ventilation solenoid valve, a ventilation solenoid valve (hereinafter referred to as a first ventilation solenoid valve) for turning ON/OFF supply of respiratory gas and a ventilation solenoid valve (hereinafter referred to as a second ventilation solenoid valve) for turning ON/OFF supply of oxygen administering gas are disposed, and the first ventilation solenoid valve and the second ventilation solenoid valve are each opened or closed at a predetermined timing.

The sternum compression means 120 is a sternum compression unit of the cardiopulmonary resuscitator 100. As illustrated in FIG. 1, the sternum compression means (hereinafter also referred to as a sternum compression unit) 120 includes an arch portion 10, a vertical rod 20, and a back plate 30.

The arch portion 10 has a top surface portion 11 and left and right side surface portions 12 and is disposed so as to extend over the chest of a patient. The arch portion 10 includes the impact hammer 121 projecting downward from the top surface portion 11 and movably supported in the vertical direction by the top surface portion 11 and an elevating means (elevating mechanism) 122 for vertically reciprocating the impact hammer 121. The impact hammer 121 includes an impact hammer rod 121a connected to the elevating means 122 and an impact head pad 121b attached to a lower end of the impact hammer rod 121a and pressed against the chest of a patient. In a case where the driving system of the elevating means 122 is a gas driving system, the elevating means 122 includes a cylinder 123. The cylinder 123 has a container shape and has a gas supply port (not illustrated) and a gas discharge port (not illustrated). A piston (not illustrated) and a spring (not illustrated) for pushing back the piston at the time of discharge are disposed in an internal space of the cylinder 123.

In the impact hammer rod 121a and the impact head pad 121b, an angle adjustment function of the impact head pad 121b is preferably disposed at a tip of the impact hammer rod 121a such that a pad surface of the impact head pad 121b can capture a compression point of the sternum of a patient all the time. The angle adjustment function has, for example, a ball joint type structure. The ball joint type structure has, for example, a structure in which a spherical ball portion (not illustrated) disposed at a tip of the impact hammer rod 121a is slidably and rotatably fitted in a housing portion (not illustrated) disposed in the impact head pad 121b. As a result, even in a case where the impact head pad 121b hits a patient obliquely, the impact head pad 121b is swingable or rotatable, and therefore the impact head pad 121b can be applied such that a pad surface thereof is parallel to the sternum of a patient all the time. As a result, fracture of a sternum can be prevented.

The driving system (not illustrated) of the elevating means 122 includes a driving gas supply source such as a gas cylinder or an air tank and piping connecting the driving gas supply source to the cylinder 123. In the middle of the piping, for example, a compression depth adjuster for adjusting the stroke width of the vertical reciprocating motion of the elevating means 122 and a compression solenoid valve for supplying driving gas into the cylinder 123 and discharging driving gas from the cylinder 123 are disposed. The driving gas supply source preferably serves also as a gas supply source of the artificial respiration unit 110. The compression solenoid valve is, for example, a three-way solenoid valve.

A part of the arch portion 10 is preferably the housing 101. The housing 101 houses the gas supply system of the first artificial respiration unit 110, the driving system for driving the elevating means 122, the control means of the cardiopulmonary resuscitator 100, and the like.

A pair of vertical rods 20 is disposed on the left and right sides and is fixed to fixing portions 13 disposed at lower ends of the left and right side surface portions 12 of the arch portion, respectively. For example, the vertical rod 20 is engaged with a ratchet of the fixing portion 13 to support the arch portion 10 so as to be movable in the vertical direction. As illustrated in FIG. 1, the vertical rod 20 preferably has a scale 21 displayed. Preferably, the arch portion 10 is pushed down toward the chest of a patient while the arch portion 10 is set on the patient, the scale is read when the impact head pad 121b comes into contact with the chest of the patient, and the read scale is recorded as the chest thickness of the patient. Based on this read chest thickness, the compression depth of the impact hammer 121 can be set. In this way, finer adjustment of the compression depth suitable for each patient can be performed.

The back plate 30 is a plate for supporting a lower surface of the chest of a patient. For example, by engaging an engagement portion (not illustrated) such as a groove or a hole formed in the back plate 30 with a projection (not illustrated) formed at a lower end of the vertical rod 20, the back plate 30 is fixed to the arch portion 10 detachably.

The control means (hereinafter also referred to as a first control unit) is, for example, a printed circuit board. The first control unit preferably controls the gas supply system of the first artificial respiration unit 110 and the driving system of the elevating means 122. The first control unit may control only one of the gas system of the first artificial respiration unit 110 and the driving system of the elevating means 122. The form in which the first control unit controls only one of the systems is, for example, a form in which the driving system of the elevating means 122 is automatically driven without being controlled by the first control unit, and the first control unit controls the gas system of the first artificial respiration unit 110 in accordance with driving of the elevating means 122.

The first control unit closes the ventilation solenoid valve in a case where a pressure detected by the respiratory tract internal pressure sensor is equal to or higher than a predetermined pressure. It is thereby possible to prevent injection of high pressure gas into a patient.

The first control unit adjusts the frequency (number of times of ventilation) of the artificial respiration cycle by the first artificial respiration unit 110. Here, the number of times of ventilation is the number of times for performing the artificial respiration cycle in one minute, for example, 6 to 20 times/minute. The flow rate (inhalation flow rate) of respiratory gas by the first artificial respiration unit 110 is a flow rate per unit time (hereinafter also referred to as a flow rate) and is fixed to, for example, 24 liters/minute. The ventilation amount of respiratory gas is a flow rate per one inhalation period, for example, 200 to 600 ml/time. The length of the inhalation period is, for example, 0.5 to 1.5 seconds. The lengths of the inhalation period and the exhalation period vary depending on the flow rate of respiratory gas, the number of times of ventilation, and the ventilation amount. For example, in a case where the flow rate of respiratory gas is 24 liters/minute, the number of times of ventilation is set to 10 times/minute, and the ventilation amount is set to 200 ml/time, the inhalation period is 0.5 seconds, and the exhalation period is 5.5 seconds.

Furthermore, the first control unit judges whether supply of oxygen administering gas is required for each recoil period and sends a supply instruction signal for administering the oxygen administering gas to the artificial respiration means 110 unless it is judged that supply of the oxygen administering gas is not required. In principle, the first control unit supplies oxygen administering gas in a recoil period other than a recoil period during which it has been judged that supply of the oxygen administering gas is not required. Preferable ranges of the flow rate and the ventilation amount of oxygen administering gas are the same as preferable ranges of the flow rate and the ventilation amount of respiratory gas. Set values of the flow rate and the ventilation amount of oxygen administering gas may be the same as set values of the flow rate and the ventilation amount of respiratory gas, or may be set separately. In a case where oxygen administering gas is supplied during a recoil period, the oxygen administering gas is preferably supplied over the entire period of the recoil period.

The first control unit judges that supply of oxygen administering gas is not required and does not send a supply instruction signal during a period under the following conditions (1) to (3) in a recoil period. In a case where the supply instruction signal is not sent, oxygen administering gas is not supplied during a recoil period.

Condition (1): the entire recoil period in a case where the entire recoil period or a part of the recoil period overlaps the inhalation period

Condition (2): the entire recoil period in a case where the inhalation period starts immediately after the recoil period

Condition (3): the entire recoil period in a case where the recoil period starts immediately after the inhalation period

In the condition (1), in a case where respiratory gas and oxygen administering gas are constituted by one gas system, one ventilation solenoid valve is shared by the respiratory gas and the oxygen administering gas, and therefore the ventilation amount is 0 ml/time (gas is not supplied) or a set ventilation amount (400 ml/time). Therefore, even if a recoil period and an inhalation period overlap with each other, the ventilation amount never exceeds the set ventilation amount. Therefore, it is possible to more easily prevent a respiratory tract internal pressure from rising excessively. In addition, in the condition (1), in a case where respiratory gas and oxygen administering gas are constituted by individual gas systems, when the oxygen administering gas is supplied during a recoil period overlapping with an inhalation period, both the ventilation solenoid valve for the respiratory gas and the ventilation solenoid valve for the oxygen administering gas are opened, and the respiratory gas and the oxygen administering gas are simultaneously supplied. As a result, the ventilation amount increases more than the set ventilation amount, and the respiratory tract internal pressure becomes too high. Therefore, by not sending a supply instruction signal, the ventilation amount is set as the set ventilation amount to prevent the respiratory tract internal pressure from rising excessively. The first control unit preferably adjusts the total flow rate of respiratory gas and oxygen administering gas supplied to a patient to a value less than a predetermined flow rate. The predetermined flow rate is a flow rate at which the ventilation amount is equal to or less than the set ventilation amount.

In the condition (2), in a case where an inhalation period starts immediately after a recoil period, when oxygen administering gas is supplied during the recoil period, the oxygen administering gas and respiratory gas are continuously supplied. As a result, the ventilation amount increases more than the set ventilation amount, and the respiratory tract internal pressure becomes too high. Therefore, by not sending a supply instruction signal, the ventilation amount is returned to the set ventilation amount to prevent the respiratory tract internal pressure from rising excessively. Here, the set ventilation amount is a ventilation amount for one time preset according to the physique or condition of a patient, and is calculated by, for example, flow rate (inhalation flow rate)×length of inhalation period. The set ventilation amount is, for example, 200, 300, 400, 500, or 600 ml/time.

In the condition (3), in a case where a recoil period starts immediately after an inhalation period, when oxygen administering gas is supplied in the recoil period, the oxygen administering gas is supplied after the inhalation and before exhalation is performed. As a result, exhalation gas cannot be discharged and the respiratory tract internal pressure becomes too high. Therefore, by not sending a supply instruction signal, the exhalation gas is discharged to prevent the respiratory tract internal pressure from rising excessively.

In a mode of supplying oxygen administering gas during a recoil period, the first control unit is preferably programmed so as not to perform oxygen administration during a recoil period performed before and after an inhalation period without passing through the entire compression period. It is possible to more reliably prevent a respiratory tract internal pressure from rising excessively. The recoil period performed before and after an inhalation period without passing through the entire compression period is, for example, (a) a recoil period in a case where an inhalation period starts immediately after the recoil period as in the condition (2), (b) a recoil period in a case where the recoil period starts immediately after an inhalation period as in the condition (3), (c) the first recoil period after end of an inhalation period in a case where the inhalation period ends during a compression period, or (d) the last recoil period before start of an inhalation period in a case where the inhalation period starts during a compression period.

Control based on the conditions (1) to (3) prioritizes gas supply for inhalation during an inhalation period over gas supply for passive oxygen inhalation during a recoil period. With such control, passive oxygen administration can be performed without impairing an effect of artificial respiration in CPR.

In the present embodiment, when the number of times of compression, the number of times of ventilation, and the ventilation amount are set, in a case where the first control unit executes a sternum compression cycle and an artificial respiration cycle based on the setting, for example, the first control unit preferably calculates a recoil period corresponding to any one of the conditions (1) to (3) in advance. Based on the calculation result, for example, the first control unit judges that supply of oxygen administering gas is not required and does not send a supply instruction signal in a recoil period corresponding to the conditions (1) to (3), and judges that supply of oxygen administering gas is required and sends the supply instruction signal in a recoil period other than the recoil period corresponding to any one of the conditions (1) to (3).

The first control unit controls the compression solenoid valve to supply driving gas into the cylinder 123 or discharge the driving gas from the inside of the cylinder 123. When the driving gas is supplied into the cylinder 123, a piston is pushed down against a repulsive force of a spring, and the impact hammer 121 moves downward. When the driving gas is discharged from the inside of the cylinder 123, the spring expands, the piston is pushed up, and the impact hammer 121 moves upward. By repeating these, the impact hammer 121 reciprocates vertically.

In addition, the first control unit adjusts the frequency of the sternum compression cycle (the number of times of compression) and the stroke width of the vertical reciprocating motion of the elevating means 122. The stroke width is switched, for example, by turning of an adjustment knob 14 by an operator. Here, the number of times of compression is the number of times for performing the sternum compression cycle in one minute, and Guidelines recommend that the number of times of compression is 100 times/minute or more. A compression period and a recoil period preferably have the same length as each other. For example, in a case where the number of times of compression is 100 times/minute, the time per sternum compression cycle is 0.6 seconds, and the compression period and the recoil period are each 0.3 seconds. The stroke width of the vertical reciprocating motion is appropriately adjusted according to a patient, but Guidelines recommend that the stroke width is 5 cm or more in an adult.

The cardiopulmonary resuscitator 100 preferably includes a mode switching unit 15. The mode switching unit 15 is a switch for switching an operation timing mode of each of the first artificial respiration unit 110 and the sternum compression unit 120. The mode switching unit 15 is, for example, a panel as illustrated in FIG. 1 or a knob (not illustrated). Although FIG. 1 illustrates an example in which the mode switching unit 15 is disposed in the arch portion 10, the present invention is not limited thereto, and the mode switching unit 15 may be disposed on the back plate 30, for example.

The operation timing mode of each of the first artificial respiration unit 110 and the sternum compression unit 120 is roughly divided into a synchronous mode and an asynchronous mode.

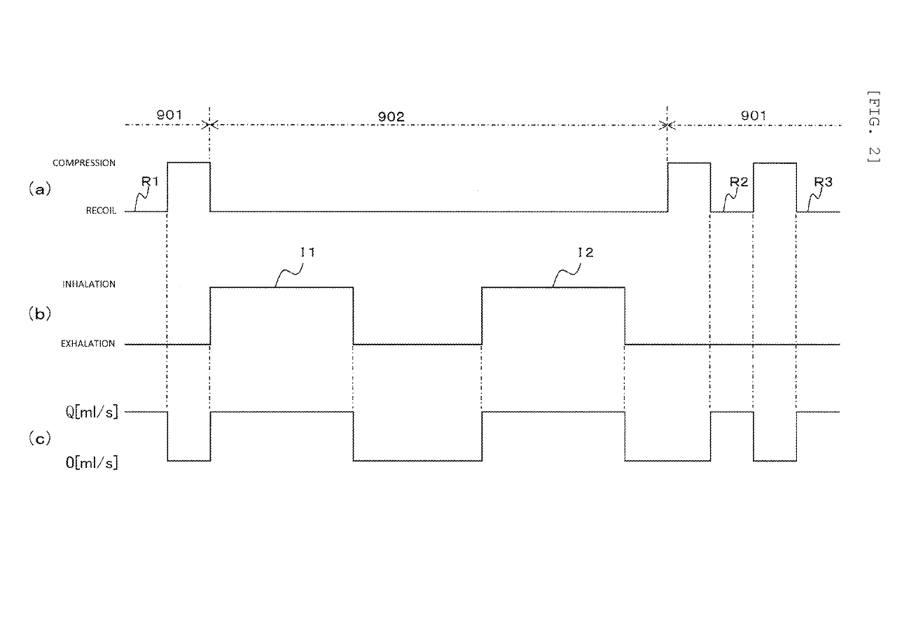

FIG. 2 is a timing chart of a sternum compression cycle and an artificial respiration cycle in a synchronous mode, FIG. 2(a) illustrates a compression waveform, FIG. 2(b) illustrates a ventilation waveform, and FIG. 2(c) illustrates a waveform of the amount of gas supplied to a patient by FIGS. 2(a) and 2(b). Here, in the timing chart, a compression waveform at the height of “compression” indicates that the compression period is being executed, and a compression waveform at the height of “recoil” indicates that the recoil period is being executed. A ventilation waveform at the height of “inhalation” indicates that the inhalation period is being executed, and a ventilation waveform at the height of “exhalation” indicates that the exhalation period is being executed. A waveform of the amount of gas at the height of 0 [ml/s] indicates that no gas is supplied to a patient, and a waveform of the amount of gas at the height of Q [ml/s] indicates that a predetermined amount of gas is supplied to a patient.

In the synchronous mode, as illustrated in FIG. 2, the first control unit causes the sternum compression unit 120 to alternately perform an execution period 901 in which the sternum compression cycle is executed a predetermined number of times per unit time and a standby period 902 in which execution of the sternum compression cycle is temporarily stopped in the state of separating the impact hammer 121 from the chest, and executes the artificial respiration cycle a predetermined number of times per unit time during the standby period 902.

In the synchronous mode, each of the recoil periods R1, R2, and R3 does not correspond to any one of the conditions (1) to (3). Therefore, the first control unit supplies oxygen administering gas during each of the recoil periods R1, R2, and R3. The first control unit supplies respiratory gas during the inhalation periods I1 and I2. With such control, as illustrated in FIG. 2(c), the waveform of the amount of gas has a shape matching the timing of each of the recoil periods R1, R2, and R3 and the inhalation periods I1 and I2. As a result, in the synchronous mode, it is possible to perform, while performing CPR, passive oxygen administration (hereinafter also referred to as “beneficial and efficient passive oxygen administration”) preventing an excessive rise in respiratory tract internal pressure and reducing oxygen consumption as compared with a conventional passive oxygen administration method.

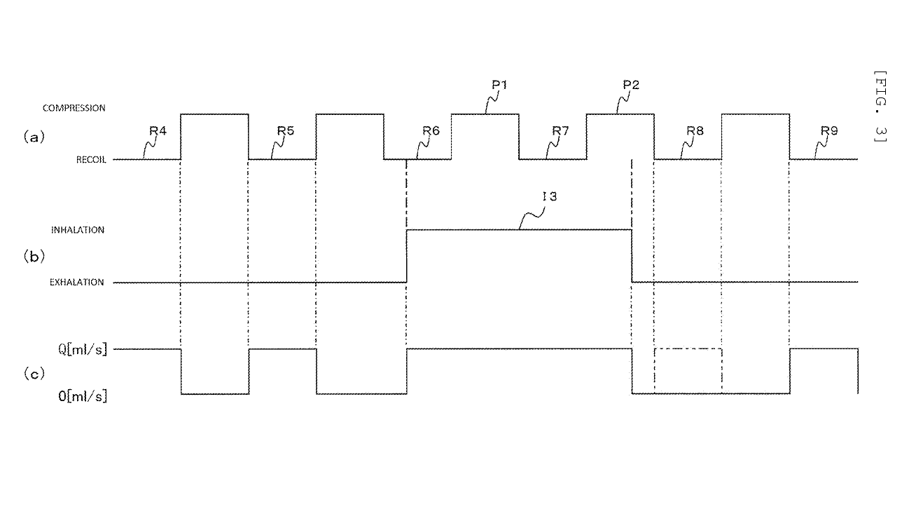

FIG. 3 is a timing chart of a sternum compression cycle and an artificial respiration cycle in an asynchronous mode, FIG. 3(a) illustrates a compression waveform, and FIG. 3(b) illustrates a ventilation waveform. In the asynchronous mode, as illustrated in FIG. 3, the first control unit executes the artificial respiration cycle a predetermined number of times per unit time while executing the sternum compression cycle a predetermined number of times per unit time.

In FIG. 3, the recoil periods R6 and R7 correspond to the condition (1). Therefore, the first control unit judges that supply of oxygen administering gas is not required and does not send a supply instruction signal in the entire recoil periods R6 and R7. Each of the other recoil periods R4, R5, R8, and R9 does not correspond to any one of the conditions (1) to (3), and therefore the first control unit supplies oxygen administering gas during each of the recoil periods R4, R5, R8, and R9. The first control unit supplies respiratory gas during the inhalation period I3. With such control, as illustrated in FIG. 3(c), the waveform of the amount of gas has a shape matching the timing of each of the recoil periods R4, R5, R8, and R9 and the inhalation period I3. As a result, it is possible to perform beneficial and efficient passive oxygen administration while performing CPR in the asynchronous mode.

As illustrated in FIG. 3, in a case where the inhalation period I3 ends during the compression period P2, preferably, the first control unit judges that supply of oxygen administering gas is not required and does not send a supply instruction signal in the first recoil period R8 after the end of the inhalation period I3 (condition (4)). The recoil period R8 starts before the gas supplied during the inhalation period I3 is sufficiently ventilated by compression. Therefore, when gas is supplied in the recoil period R8, gas is further supplied before the respiratory tract internal pressure sufficiently drops, and the respiratory tract internal pressure may be too high. Therefore, by not sending a supply instruction signal in the first recoil period R8 performed after the inhalation period I3, it is possible to more reliably prevent an excessive rise in respiratory tract internal pressure.

In a case where the respiratory gas and the oxygen administering gas have the same composition, a case is considered in which it cannot be judged whether the gas supply of which is stopped during the recoil periods R6 and R7 is the oxygen administering gas or the respiratory gas. In this case, as illustrated in FIG. 3(c), the waveform of the amount of gas for a predetermined time is observed, and if the waveform of the amount of gas matches the timing of each of the recoil periods R4, R5, R8, and R9 and the inhalation period I3, it is judged that supply of oxygen administering gas is not required during the recoil periods R6 and R7, that is, it is regarded that a supply instruction signal has not been sent. The waveform of the amount of gas observed is preferably, for example, a waveform for one to five minutes.

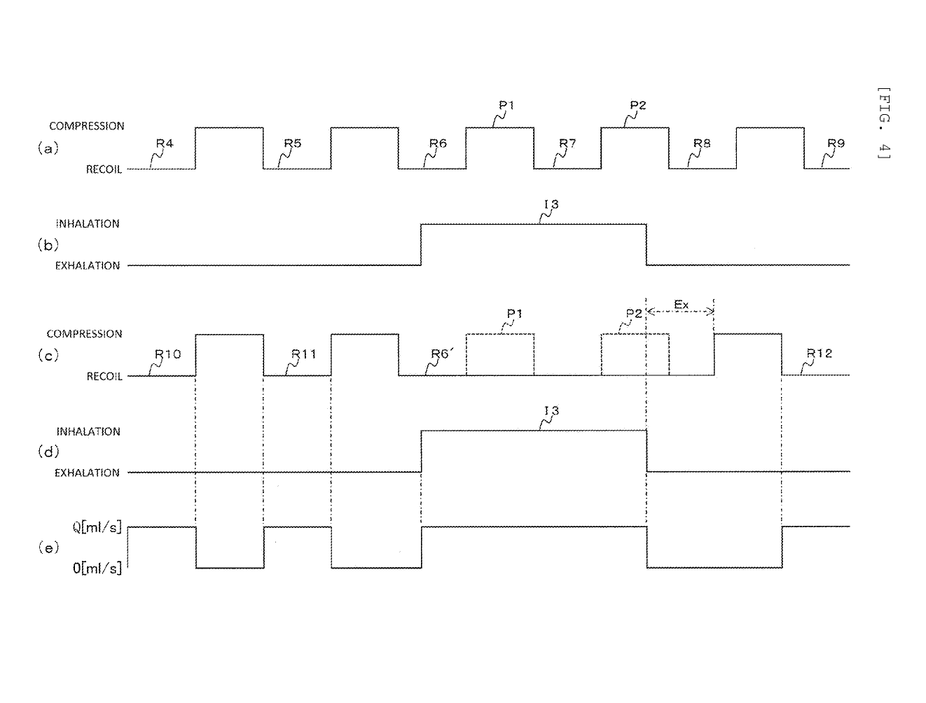

In FIGS. 4(a) and 4(b), the entire compression period P1 and a part of the compression period P2 overlap with the inhalation period I3. In the overlapping periods, a phenomenon (also referred to as fighting) in which the timing of sternum compression and the timing of artificial respiration are coincident and exhalation of a patient and inhalation by an artificial respirator occur simultaneously occurs, and a respiratory tract internal pressure instantaneously rises. When the pressure detected by the respiratory tract internal pressure sensor reaches a predetermined pressure or more, a ventilation solenoid valve is closed and an inhalation period may end forcibly. In the cardiopulmonary resuscitation system according to the present embodiment, in the compression periods P1 and P2 overlapping with the inhalation period I3, the first control unit preferably stops pressing the impact hammer 121 against the chest. This can avoid fighting. As a specific example of avoiding fighting, in a case where the sternum compression cycle and the artificial respiration cycle are executed based on setting of the number of times of compression, the number of times of ventilation, and the ventilation amount, the first control unit calculates a compression period overlapping with an inhalation period in advance and, for example, as illustrated in FIGS. 4(c) and 4(d), shifts the timing of the compression period or the timing of the inhalation period such that the inhalation period and the compression period do not overlap with each other. Here, the asynchronous mode in the present invention for avoiding fighting may be referred to as an “avoidance type asynchronous mode” in order to distinguish the asynchronous mode in the present invention from a conventional asynchronous mode in which fighting occurs.

The avoidance type asynchronous mode is roughly divided into three patterns depending on whether the start time of the inhalation period is during the recoil period (first example), during the first half period of the compression period (second example), or during the second half period of the compression period (third example). Next, with reference to FIGS. 4 to 6, specific examples of avoiding fighting and passive oxygen administration in each of the examples will be described.

### First Example

FIGS. 4(a) and 4(b) are waveforms in a case where the sternum compression cycle and the artificial respiration cycle are executed based on setting of the number of times of compression, the number of times of ventilation, and the length of the inhalation period. In FIGS. 4(a) and 4(b), the inhalation period I3 is started during the recoil period R6. In this state, the entire compression period P1 and a part of the compression period P2 overlap with the inhalation period I3. In this case, as illustrated in FIGS. 4(c) and 4(d), the first control unit extends the recoil period R6′ executed at the start time of the inhalation period I3 at least until the inhalation period I3 ends. As a result, overlapping between the inhalation period I3 and the compression periods P1 and P2 is eliminated, and fighting can be avoided. In a case where the entire compression period P1 overlaps with the inhalation period I3, the first control unit cancels the entire compression period P1 (indicated by a dotted line in FIG. 4(c)). In a case where a part of the compression period P2 overlaps with the inhalation period I3, the first control unit preferably cancels the entire compression period P2 (indicated by a dotted line in FIG. 4(c)). Alternatively, the first control unit may cancel only a period overlapping with the inhalation period I3 in the compression period P2. In the first example, the number of times of compression is smaller than a set value by the amount overlapping with the inhalation period, and the number of times of ventilation is the same as a set value.

In FIGS. 4(c) and 4(d), the extended recoil period R6′ corresponds to the condition (1). Therefore, the first control unit judges that supply of oxygen administering gas is not required and does not send a supply instruction signal in the entire recoil period R6′. Each of the other recoil periods R10, R11, and R12 does not correspond to any one of the conditions (1) to (3), and therefore the first control unit supplies oxygen administering gas during each of the recoil periods R10, R11, and R12. The first control unit supplies respiratory gas during the inhalation period I3. With such control, as illustrated in FIG. 4(e), the waveform of the amount of gas has a shape matching the timing of each of the recoil periods R10, R11, and R12 and the inhalation period I3. As a result, it is possible to perform beneficial and efficient passive oxygen administration while performing CPR in the avoidance type asynchronous mode.

### Second Example

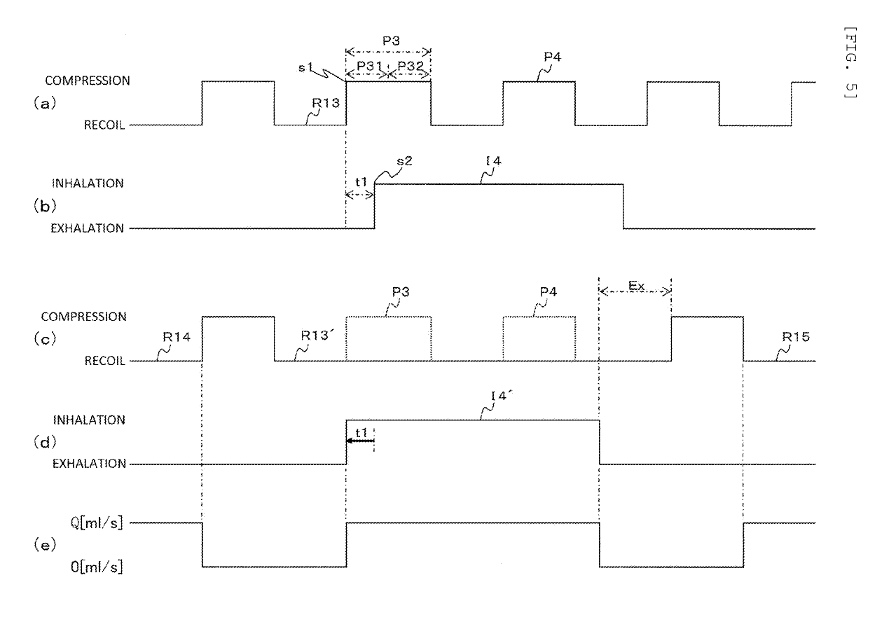

FIGS. 5(a) and 5(b) are waveforms in a case where the sternum compression cycle and the artificial respiration cycle are executed based on setting of the number of times of compression, the number of times of ventilation, and the length of the inhalation period. In FIGS. 5(a) and 5(b), the inhalation period I4 is started during the compression period P3, and the start time s2 of the inhalation period I4 is during the first half period P31 obtained by temporally dividing the compression period P3 into two equal parts. In this state, a part of the compression period P3 and the entire compression period P4 overlap with the inhalation period I4. In this case, as illustrated in FIGS. 5(c) and 5(d), the first control unit preferably hastens start of the inhalation period I4′ by the same time as the time t1 from the start time s1 of the compression period P3 overlapping with the inhalation period I4 to the start time s2 of the inhalation period I4. At this time, the first control unit preferably cancels the entire compression periods P3 and P4 overlapping with the inhalation period I4′ which has been started earlier (indicated by a dotted line in FIG. 5(c)). The first control unit extends the recoil period R13 immediately before the compression period P3 to set the recoil period R13′. As a result, fighting can be avoided even in a case where the inhalation period I4 is started during the compression period P3.

The length of the exhalation period I4′ which has been started earlier is the same as the length of the exhalation period I4 which has been scheduled to be executed before the start time is made earlier. That is, the exhalation period I4′ is temporally shifted forward by the time t1 with respect to the exhalation period I4. In the second example, the number of times of compression is smaller than a set value by the amount overlapping with the inhalation period, and the number of times of ventilation fluctuates with respect to a set value by the amount of shift. The variation in the number of times of ventilation is preferably within ±5% of a set value.

In FIGS. 5(c) and 5(d), the extended recoil period R13′ corresponds to the condition (1). Therefore, the first control unit judges that supply of oxygen administering gas is not required and does not send a supply instruction signal in the entire recoil period R13′. Each of the other recoil periods R14 and R15 does not correspond to any one of the conditions (1) to (3), and therefore the first control unit supplies oxygen administering gas during each of the recoil periods R14 and R15. The first control unit supplies respiratory gas during the inhalation period I4′. With such control, as illustrated in FIG. 5(e), the waveform of the amount of gas has a shape matching the timing of each of the recoil periods R14 and R15 and the inhalation period I4′. As a result, it is possible to perform beneficial and efficient passive oxygen administration while performing CPR in the avoidance type asynchronous mode.

As illustrated in FIGS. 5(a) and 5(b), in a case where the inhalation period I4 starts during the compression period P3, preferably, the first control unit judges that supply of oxygen administering gas is not required and does not send a supply instruction signal in the last recoil period R13 before the start of the inhalation period I4 (condition (5)). It is possible to more reliably prevent an excessive rise in respiratory tract internal pressure.

### Third Example

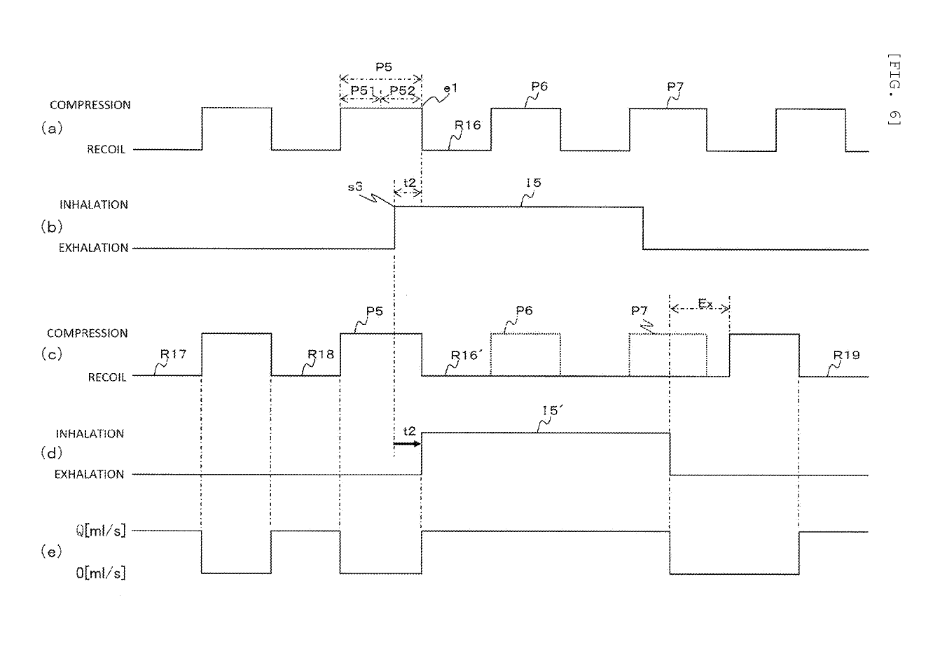

FIGS. 6(a) and 6(b) are waveforms in a case where the sternum compression cycle and the artificial respiration cycle are executed based on setting of the number of times of compression, the number of times of ventilation, and the length of the inhalation period. In FIGS. 6(a) and 6(b), the inhalation period I5 is started during the compression period P5, and the start time s3 of the inhalation period I5 is during the second half period P52 obtained by temporally dividing the compression period P5 into two equal parts. In this state, a part of the compression periods P5 and P7 and the entire compression period P6 overlap with the inhalation period I5. In this case, as illustrated in FIGS. 6(c) and 6(d), the first control unit preferably delays start of the inhalation period I5′ by the same time as the time t2 from the start time s3 of the inhalation period I5 to the end time e1 of the compression period P5 overlapping with the inhalation period I5. At this time, the inhalation period I5′ is started after the end of the compression period P5, and the compression period P5 is executed because the compression period P5 does not overlap with the inhalation period I5′ the start of which has been delayed. The first control unit preferably cancels the entire compression periods P6 and P7 overlapping with the inhalation period I5′ the start of which has been delayed (indicated by a dotted line in FIG. 6(c)). The first control unit extends the recoil period R16 immediately before the compression period P6 to set the recoil period R16′. As a result, fighting can be avoided even in a case where the inhalation period I5 is started during the compression period P5.

The length of the exhalation period I5′ the start of which has been delayed is the same as the length of the exhalation period I5 which has been scheduled to be executed before the start is delayed. That is, the exhalation period I5′ is temporally shifted backward by the time t2 with respect to the exhalation period I5. In the third example, the number of times of compression is smaller than a set value by the amount overlapping with the inhalation period, and the number of times of ventilation fluctuates with respect to a set value by the amount of shift. The variation in the number of times of ventilation is preferably within ±5% of a set value.

In FIGS. 6(c) and 6(d), the extended recoil period R16′ corresponds to the condition (1). Therefore, the first control unit judges that supply of oxygen administering gas is not required and does not send a supply instruction signal in the entire recoil period R16′. Each of the other recoil periods R17, R18, and R19 does not correspond to any one of the conditions (1) to (3), and therefore the first control unit supplies oxygen administering gas during each of the recoil periods R17, R18, and R19. The first control unit supplies respiratory gas during the inhalation period I5′. With such control, as illustrated in FIG. 6(e), the waveform of the amount of gas has a shape matching the timing of each of the recoil periods R17, R18, and R19 and the inhalation period I5′. As a result, it is possible to perform beneficial and efficient passive oxygen administration while performing CPR in the avoidance type asynchronous mode.

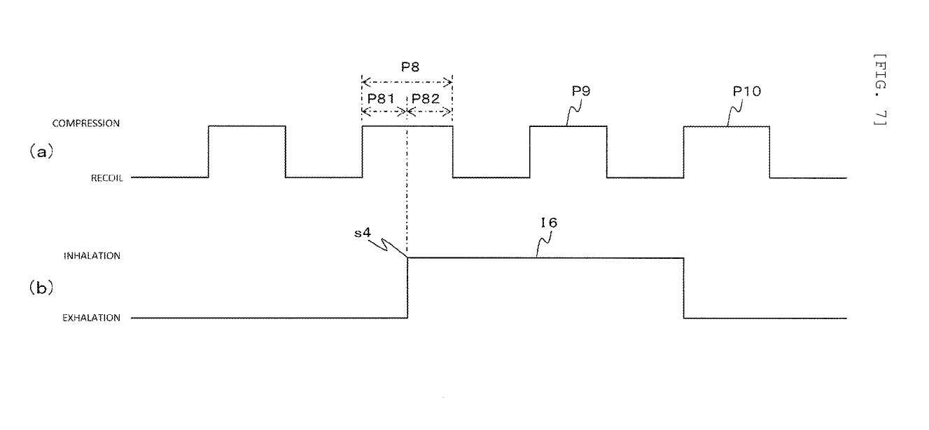

As illustrated in FIGS. 7(a) and 7(b), in a case where the inhalation period I6 is started during the compression period P8 and the start time s4 of the inhalation period I6 is at a boundary between the first half period P81 and the second half period P82 obtained by temporally dividing the compression period P8 into two equal parts, the first control unit may shift the inhalation period I6 forward as in the second example to cancel the entire compression periods P8 and P9 overlapping with the inhalation period (not illustrated) which has been shifted forward, or may shift the inhalation period I6 backward as in the third example to cancel the entire compression periods P9 and P10 overlapping with the inhalation period (not illustrated) which has been shifted backward.

In the first to third examples, as illustrated in FIGS. 4(c) and 4(d), FIGS. 5(c) and 5(d), and FIGS. 6(c) and 6(d), the first control unit preferably restarts the sternum compression cycle a predetermined time after the end of each of the inhalation periods I3, I4′, and I5′, and the sternum compression cycle restarted is preferably started from the compression period. In other words, the first control unit preferably ends the extended recoil periods R6′, R13′, and R16′ after the inhalation periods I3, I4′, and I5′ are ended, respectively. By setting delay time Ex during which the impact hammer 121 is separated from the chest after the inhalation periods I3, I4′, and I5′ are ended, exhalation time can be secured to prevent a respiratory tract internal pressure from becoming too high. The length of the delay time Ex is not particularly limited, and for example, as compared with a recoil period determined based on setting of the number of times of ventilation and the length of the inhalation period, the length of the delay time Ex may be the same as the length of the recoil period, may be longer or shorter than the length of the recoil period, or may be longer than the length of the recoil period.

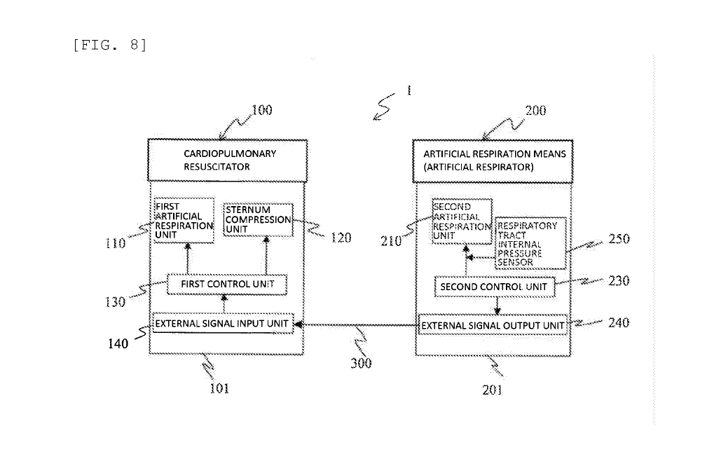

FIG. 8 is a conceptual diagram of another example of the cardiopulmonary resuscitation system according to the present embodiment. Up to this point, the form in which the artificial respiration means (first artificial respiration unit) 110 and the sternum compression means (sternum compression unit) 120 are formed into an integral device as illustrated in FIG. 1 has been described as an example, but the present embodiment is not limited thereto. In a cardiopulmonary resuscitation system 1 according to the present embodiment, as illustrated in FIG. 8, the artificial respiration means 200 and the sternum compression means 120 may be configured as individual devices. By using the artificial respiration means 200 and the sternum compression means 120 as individual device, more accurate cardiopulmonary resuscitation can be performed.

In FIG. 8, the artificial respiration means 200 is an artificial respirator. An artificial respiration means (hereinafter also referred to as an artificial respirator) 200 is, for example, an artificial respirator ANSWER for emergency transport (registered trademark) (manufactured by Kohken Medical Co., Ltd.). As illustrated in FIG. 8, the artificial respirator 200 includes: a second artificial respiration unit 210 for injecting respiratory gas or oxygen administering gas into a patient; a second control unit 230 for controlling the second artificial respiration unit 210 and generating an external signal including a remote control signal to a cardiopulmonary resuscitator; an external signal output unit 240 for outputting the external signal generated by the second control unit 230 to an outside; a respiratory tract internal pressure sensor 250 for detecting a respiratory tract internal pressure of a patient; and a housing 201 for housing these.

The second artificial respiration unit 210 includes an inhalation hose (not illustrated) for injecting respiratory gas or oxygen administering gas into a patient and a gas supply system (not illustrated) for supplying respiratory gas or oxygen administering gas. One end of the inhalation hose is connected to a hose insertion port (not illustrated) disposed in the housing 201 of the artificial respirator 200. The other end of the inhalation hose is connected to a mask (not illustrated) attached to a patient or a tracheal intubation tube (not illustrated). The gas supply system of the second artificial respiration unit 210 has the same basic configuration as the gas supply system of the first artificial respiration unit 110. A major difference is that a ventilation solenoid valve is disposed in the middle of the piping in the first artificial respiration unit 110, whereas a flow-controllable valve such as a flow regulating valve is disposed in the middle of the piping in the second artificial respiration unit 210.

The second control unit 230 is, for example, a printed circuit board. The second control unit 230 controls the gas supply system of the second artificial respiration unit 210. The second control unit 230 generates an external signal.

The second control unit 230 adjusts the frequency (number of times of ventilation) of the artificial respiration cycle by the second artificial respiration unit 210. Here, the number of times of ventilation is, for example, 2 to 40 times/minute. The second control unit 230 adjusts the ventilation amount of respiratory gas or oxygen administering gas and the length of an inhalation period. The first artificial respiration unit 110 includes a ventilation solenoid valve, whereas the second artificial respiration unit 210 includes a flow-controllable valve such as a flow regulating valve. Therefore, the ventilation amount of respiratory gas or oxygen administering gas by the second artificial respiration unit 210 can be adjusted in a wider range than the range of the ventilation amount of respiratory gas or oxygen administering gas by the first artificial respiration unit 110, and is, for example, 50 to 3000 ml/time. The inhalation period of respiratory gas or oxygen administering gas by the first artificial respiration unit 110 is automatically switched according to the flow rate of respiratory gas or oxygen administering gas, the number of times of ventilation, and the ventilation amount, whereas the inhalation period of respiratory gas or oxygen administering gas by the second artificial respiration unit 210 can be continuously switched alone, for example within a range of 0.3 to 3.0 seconds. Therefore, the second artificial respiration unit 210 can adjust the flow rate more finely than the first artificial respiration unit 110.

The external signal output unit 240 is, for example, a connection terminal of a cable (not illustrated) or a transmission unit of a wireless signal or the like, and outputs an external signal sent from the second control unit 230.

The respiratory tract internal pressure sensor 250 can detect from a negative pressure to a positive pressure, detects a respiratory tract internal pressure of a patient, and outputs a pressure signal to the second control unit 230.

In FIG. 8, the sternum compression means 120 is a sternum compression unit of the cardiopulmonary resuscitator 100. The cardiopulmonary resuscitator 100 has the same basic configuration as the cardiopulmonary resuscitator illustrated in FIG. 1, for example. In a case where the cardiopulmonary resuscitator 100 cooperates with the artificial respirator 200, as illustrated in FIG. 1, the cardiopulmonary resuscitator 100 preferably includes an external signal input unit 140 for inputting an external signal including a remote control signal instructing the sternum compression unit 120 to perform sternum compression. The external signal input unit 140 is, for example, a connection terminal of a cable (not illustrated) or a reception unit of a wireless signal or the like. An external signal input from the external signal input unit 140 is sent to a first control unit 130. The cardiopulmonary resuscitator 100 and the artificial respirator 200 are connected to each other such that an external signal can be transmitted by a signal transmission means 300. The signal transmission means 300 is, for example, a connection cable or wireless communication. The signal transmission means 300 transmits an external signal from the external signal output unit 240 to the external signal input unit 140.

In the cardiopulmonary resuscitation system 1, the artificial respirator 200 performs artificial respiration, and the cardiopulmonary resuscitator 100 performs only sternum compression. That is, the sternum compression unit 120 and the second artificial respiration unit 210 are in an operable state, and the first artificial respiration unit 110 is in a stopped state. At this time, the signal transmission means 300 makes it possible to transmit an external signal from the artificial respirator 200 to the cardiopulmonary resuscitator 100.

In the cardiopulmonary resuscitation system 1, a control means for controlling the artificial respiration means (artificial respirator) 200 and the sternum compression means (sternum compression unit) 120 are preferably mounted on the artificial respirator 200. By control of the sternum compression unit 120 in addition to the second artificial respiration unit 210 by the second control unit 230, the cardiopulmonary resuscitator 100 can be small and lightweight, and sternum compression can be quickly started in an early stage of critical care. The second control unit 230 generates, for example, an external signal including a remote control signal instructing the sternum compression unit 120 to perform sternum compression. The external signal including the remote control signal is output from the external signal output unit 240 and input to the external signal input unit 140 by the signal transmission means 300. The external signal including the remote control signal input by the external signal input unit 140 is sent to the first control unit 130. Upon input of the remote control signal, the first control unit 130 drives the sternum compression unit 120. In this way, the second control unit 230 remotely controls the sternum compression unit 120. In a case where the pressure detected by the respiratory tract internal pressure sensor 250 is a negative pressure, the second control unit 230 outputs a signal for opening a flow regulating valve (not illustrated) to the flow regulating valve (not illustrated) and supplies respiratory gas or oxygen administering gas from the second artificial respiration unit.

Passive oxygen administration by the second control unit 230 is performed in a similar manner to those in FIGS. 4 to 6.

Up to this point, the form in which the elevating means 122 of the sternum compression means 120 is a gas driving system has been described, but the present invention is not limited to the driving system of the elevating means 122. The driving system of the elevating means 122 may be, for example, an electric driving system or a mixed driving system of a gas driving system and an electric driving system. In a case where the driving system of the elevating means 122 is a gas driving system, according to the classification of the medical apparatus law or the like, the sternum compression means 120 may also be referred to as a “mechanical cardiopulmonary artificial resuscitator”. By adopting the gas driving system, the setting width of the sternum compression depth can be wide, and sternum compression by a method with hands can be reproduced. In a case where the driving system of the elevating means 122 is an electric driving system, according to the classification of the medical apparatus law or the like, the sternum compression means 120 may also be referred to as an “electric cardiopulmonary artificial resuscitator”. By adopting the electric driving system, a device can be simpler. The electric driving system is, for example, an internal battery, an external battery, an external power source such as an AC 100 V power source, or a combination thereof. In a case where the electric driving system is adopted as the driving system of the elevating means 122, the impact hammer 121 reciprocates vertically by motor driving, for example.

The cardiopulmonary resuscitation system according to the present embodiment may have a mode of not supplying oxygen administering gas during a recoil period in addition to a principle mode of supplying oxygen administering gas during a recoil period. The modes can be switched therebetween according to the state of a patient.

### REFERENCE SIGNS LIST

1, 100 Cardiopulmonary resuscitation system
10 Arch portion
11 Top surface portion
12 Left and right side surface portions
13 Fixing portion
14 Adjustment knob
15 Mode switching unit
20 Vertical rod
21 Scale
30 Back plate
101 Housing
110 Artificial respiration means (first artificial respiration unit)
111 Hose
112 Hose insertion port
120 Sternum compression means
121 Impact hammer
121a Impact hammer rod
121b Impact head pad
122 Elevating means
123 Cylinder
130 First control unit
140 External signal input unit
200 Artificial respiration means (artificial respirator)
201 Housing
210 Second artificial respiration unit
230 Second control unit
240 External signal output unit
250 Respiratory tract internal pressure sensor
300 Signal transmission unit
901 Execution period
902 Standby period
R1, R2, R3, R4, R5, R6, R6′, R7, R8, R9, R10, R11, R12, R13, R13′, R14, R15, R16, R16′, R17, R18, and R19 Recoil period
I1, I2, I3, I4, I4′, I5, I5′, and I6 Inhalation period
P1, P2, P3, P4, P5, P6, P7, P8, P9, and P10 Compression period
P31 First half period
P32 Second half period
P51 First half period
P52 Second half period
P81 First half period
P82 Second half period

What is claimed is:

1. A cardiopulmonary resuscitation system comprising:
a sternum compression unit that includes an impact hammer for compressing the chest of a patient and repeats a sternum compression cycle having, as one cycle, a compression period in which the impact hammer is pressed against the chest and a recoil period in which the impact hammer is separated from the chest and which be performed succeeding to the compression period;
an artificial respiration unit that repeats an artificial respiration cycle having, as one cycle, an inhalation period in which respiratory gas is supplied to the patient and an exhalation period in which supply of the respiratory gas is stopped and can supply oxygen administering gas for passive oxygen inhalation to the patient during the recoil period; and
a controller that controls the artificial respiration unit and/or the sternum compression unit, wherein
the control controller judges, for each recoil period, whether supply of the oxygen administering gas is required and sends a supply instruction signal to administer the oxygen administering gas to the artificial respiration unit unless it is judged that supply of the oxygen administering gas is not required.

2. The cardiopulmonary resuscitation system according to claim 1, wherein in a period under the following conditions (1) to (3), the controller judges that supply of the oxygen administering gas is not required and does not send the supply instruction signal, wherein the conditions are as follows:
Condition (1): an entire recoil period in a case where the entire recoil period or a part of the recoil period overlaps the inhalation period;
Condition (2): the entire recoil period in a case where the inhalation period starts immediately after the recoil period; and
Condition (3): the entire recoil period in a case where the recoil period starts immediately after the inhalation period.

3. The cardiopulmonary resuscitation system according to claim 1, wherein the controller causes the sternum compression unit to alternately perform an execution period in which the sternum compression cycle is executed a predetermined number of times per unit time and a standby period in which execution of the sternum compression cycle is temporarily stopped in the state of separating the impact hammer from the chest, and executes the artificial respiration cycle a predetermined number of times per unit time during the standby period.

4. The cardiopulmonary resuscitation system according to claim 1, wherein the controller executes the artificial respiration cycle a predetermined number of times per unit time while executing the sternum compression cycle a predetermined number of times per unit time.

5. The cardiopulmonary resuscitation system according to claim 4, wherein
in a case where the inhalation period ends during the compression period,
in the first recoil period after the end of the inhalation period, the controller judges that supply of the oxygen administering gas is not required and does not send the supply instruction signal.

6. The cardiopulmonary resuscitation system according to claim 4, wherein
in a case where the inhalation period starts during the compression period,
in the last recoil period before the start of the inhalation period, the controller judges that supply of the oxygen administering gas is not required and does not send the supply instruction signal.

7. The cardiopulmonary resuscitation system according to claim 4, wherein in the compression period overlapping with the inhalation period, the controller stops pressing the impact hammer against the chest.

8. The cardiopulmonary resuscitation system according to claim 7, wherein
in a case where the inhalation period is started during the recoil period,
the controller extends the recoil period executed at the start time of the inhalation period at least until the inhalation period ends.

9. The cardiopulmonary resuscitation system according to claim 7, wherein
in a case where the inhalation period is started during the compression period and the start time of the inhalation period is in the first half period obtained by temporally dividing the compression period into two equal parts,
the controller hastens start of the inhalation period by the same time as the time from the start time of the compression period overlapping with the inhalation period to the start time of the inhalation period.

10. The cardiopulmonary resuscitation system according to claim 7, wherein
in a case where the inhalation period is started during the compression period and the start time of the inhalation period is in the second half period obtained by temporally dividing the compression period into two equal parts,
the controller delays start of the inhalation period by the same time as the time from the start time of the inhalation period to the end time of the compression period overlapping with the inhalation period.

11. The cardiopulmonary resuscitation system according to claim 7, wherein
the controller restarts the sternum compression cycle a predetermined time after the end of the inhalation period, and
the sternum compression cycle restarted is started from the compression period.

12. The cardiopulmonary resuscitation system according to claim 1, wherein
the artificial respiration unit and the sternum compression unit are configured as an integral device.

13. The cardiopulmonary resuscitation system according to claim 1, wherein
the artificial respiration unit and the sternum compression unit are configured as individual devices.

14. The cardiopulmonary resuscitation system according to claim 13, wherein the controller is mounted on a device constituting the artificial respiration unit.

15. The cardiopulmonary resuscitation system according to claim 8, wherein
the controller restarts the sternum compression cycle a predetermined time after the end of the inhalation period, and
the sternum compression cycle restarted is started from the compression period.

16. The cardiopulmonary resuscitation system according to claim 9, wherein
the controller restarts the sternum compression cycle a predetermined time after the end of the inhalation period, and
the sternum compression cycle restarted is started from the compression period.

17. The cardiopulmonary resuscitation system according to claim 10, wherein
the controller restarts the sternum compression cycle a predetermined time after the end of the inhalation period, and
the sternum compression cycle restarted is started from the compression period.
